(19) United States
(12) Patent Application Publication
(10) Pub. No.: US 2024/0033077 A1
(43) Pub. Date: Feb. 1, 2024

(54) Foldable Prosthetic Heart Valve

(71) Applicant: The Medical College of Wisconsin, Inc., Milwaukee, WI (US)

(72) Inventors: Brandon James Tefft, Menomonee Falls, WI (US); Yang Wei William Yuan, Wauwatosa, WI (US)

(21) Appl. No.: 18/247,673

(22) PCT Filed: Oct. 1, 2021
(86) PCT No.: PCT/US2021/053164

§ 371 Date: Apr. 3, 2023

Related U.S. Application Data
(60) Provisional application No. 63/086,564, filed on Oct. 1, 2020.

(51) Int. Cl. A61F 2/24

(52) U.S. Cl. CPC A61F 2/2418; A61F 2230/0023; A61F 2230/0067

(57) ABSTRACT

A prosthetic heart valve is constructed from a flexible substrate that can be folded from an unfolded configuration to a folded configuration, in which the prosthesis is operable as a prosthetic heart valve. The prosthetic heart valves can be designed as atrioventricular valves (e.g., tricuspid valve, mitral valve) or as semilunar valves (e.g., aortic valve, pulmonary valve).

### CROSS-REFERENCE TO RELATED APPLICATIONS

This application claims the benefit of U.S. Provisional Patent Application Ser. No. 63/086,564, filed on Oct. 1, 2020, and entitled “FOLDABLE PROSTHETIC HEART VALVE,” which is herein incorporated by reference in its entirety.

### BACKGROUND

Heart valve disease affects upwards of 5 million Americans. Annually, 150,000 heart valve transplants are performed using either a mechanical valve or a bovine or porcine xenograft. Mechanical valve recipients have a lifespan of 20-30 years and require lifelong anticoagulant therapy. Xenografts are treated to prevent transplant rejection, limiting their lifespan to 20 years. Currently, many xenograft-based prosthetic heart valves are made using a handsewn construction. This process is time-consuming and costly.

The ideal transplant is a living valve made from the patient's own tissues. Researchers are attempting to grow cells into a valve shape using an extracellular matrix (“ECM”) scaffold, but to date none are fully functional.

Thus, there is a need to develop a method for valve fabrication that is simpler in construction than using ECM scaffolds, but still allows for flexible sheets of material to be used.

### SUMMARY OF THE DISCLOSURE

The present disclosure addresses the aforementioned drawbacks by providing a prosthetic heart valve that is composed from a flexible substrate having a plurality of leaflet-defining regions and a plurality of outer wall-defining regions. When the flexible membrane is folded into a folded configuration the plurality of outer wall-defining regions form a cylindrical outer wall and the plurality of leaflet-defining regions form a corresponding plurality of flexible leaflets arranged within a lumen of the cylindrical outer wall.

It is another aspect of the present disclosure to provide a prosthetic heart valve device that includes a flexible substrate having an annular shape, where the flexible substrate includes an outer periphery that circumscribes the flexible substrate, an inner periphery that defines an aperture of the annular shape, a plurality of leaflet-defining regions, and a plurality of wall-defining regions. Each leaflet-defining region is defined by an arcuate segment extending along the outer periphery from a first point to a second point, a first edge line extending from the first point to a nadir point on the inner periphery, and a second edge line extending from the second point to the nadir point on the inner periphery. Each wall-defining region is defined as a portion of the flexible substrate opposed by the first edge line of a first leaflet-defining region and the second edge line of a second leaflet-defining region that is adjacent the first leaflet-defining region. When in a folded configuration, the first edge line and the second edge line of each leaflet-defining portion meet in a seam region such that the plurality of wall-defining regions circumscribe a cylindrical volume and define an outer wall, and such that each leaflet-defining region extends from its nadir point at an inflow end of cylindrical volume to at least a free edge lying in a transverse plane at an outflow end of the cylindrical volume.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is an isometric view of a prosthetic heart valve according to some embodiments described in the present disclosure, where the valve is shown in a closed state.

FIG. 2 is an isometric view of a prosthetic heart valve according to some embodiments described in the present disclosure, where the valve is shown in an open state.

FIG. 3 is a top view of a prosthetic heart valve according to some embodiments described in the present disclosure, where the valve is shown in a closed state.

FIG. 4 is a top view of a prosthetic heart valve according to some embodiments described in the present disclosure, where the valve is shown in an open state.

FIG. 5 shows an example of a flat flexible substrate that can be folded into a folded configuration that defines a prosthetic heart valve.

FIG. 6 shows an example of a curved flexible substrate that can be folded into a folded configuration that defines a prosthetic heart valve.

FIG. 7 is a cross-sectional view of a prosthetic heart valve in its closed state, showing the folded construction of its leaflet portions and outer wall portions, where the cross-sectional view is taken in a plane parallel to the longitudinal axis of the prosthetic heart valve.

FIG. 8 is another cross-sectional view of a prosthetic heart valve in its closed state, showing the folded construction of its leaflet portions and outer wall portions, where the cross-sectional view is taken in a plane transverse to the longitudinal axis of the prosthetic heart valve.

FIGS. 9A-9D show an example of folding a flat flexible substrate into a prosthetic heart valve according to some embodiments described in the present disclosure. FIG. 9A shows the initial pattern, FIG. 9B and FIG. 9C show transitional stages of the folding process, and FIG. 9D is the final valve.

FIG. 10 shows an example of folding a flat flexible substrate into an initial folded configuration where the leaflet portions are located exterior to the central lumen of the prosthetic heart valve, and then inverting the initial folded configuration to create the final folded configuration where the leaflet portions are located within the central lumen of the prosthetic heart valve.

FIG. 11 shows an example of a flat flexible substrate that is fabricated by cutting a shape from a sheet of material, where the shape corresponds to a discontinuous annular shape that is discontinuous at two cut edges that when joined or otherwise coupled will form a curved flexible substrate.

FIG. 12 shows an example of a curved flexible substrate formed on a conical shaped mold, which may be a capture surface, a casting mold, or the like.

FIG. 13 shows an example of a conical shaped mold created using an additive manufacturing process.

FIG. 14 shows an example of a flexible substrate having a conical annular shape.

FIG. 15 illustrates how folding the flexible substrate of FIG. 14 can result in a prosthetic heart valve having three leaflet portions that are flexible between a closed state and an open state.

FIG. 16 shows an example of a flexible substrate having a conical annular shape and a tubular portion that extends outward from the tip of the conical annular shape such that the tubular portion can be inverted over the conical annular shape when in the folded configuration in order to form an outer wall, or frame, of the prosthetic heart valve.

FIG. 17 shows an example setup for fabricating a curved (or flat) flexible substrate using electrospun polyurethane.

FIG. 18 shows an example of an electrospun polyurethane flexible substrate.

### DETAILED DESCRIPTION

Described here are prosthetic heart valves that can be designed to replace damaged or diseased native heart valves. Advantageously, the prosthetic heart valves are constructed from a flexible substrate that can be folded from an unfolded configuration to a folded configuration, in which the prosthesis is operable as a prosthetic heart valve. The prosthetic heart valves can be designed as atrioventricular valves (e.g., tricuspid valve, mitral valve) or as semilunar valves (e.g., aortic valve, pulmonary valve).

In general, the prosthetic heart valves described in the present disclosure are constructed from a single flexible substrate that is folded to create a prosthetic heart valve. A suitable flexible substrate can start in one of two forms: a flat two-dimensional sheet, or a three-dimensional shape (e.g., a curved sheet). As one example, the three-dimensional shape may be similar to a hollow conical frustum. In the case of a flat sheet, a specific shape can be cut from the sheet, folded around, and the ends secured in a way that produces the three-dimensional shape. Alternatively, the three-dimensional shape can be fabricated directly.

The three-dimensional shape is then folded in a manner that produces two or three leaflets within a hollow cylinder, similar to the configuration of the leaflets and root of a native heart valve. In some configurations, the two-dimensional shape may be directly folded to form the prosthetic heart valve. In either instance, the folded shape can be maintained by attaching adjacent portions of the material by any suitable means (e.g. sutures). The valve can be implanted surgically (with or without a support frame) or affixed to a stent and implanted via a catheter. In some embodiments, a support frame can be provided at the inflow end 12 of the prosthetic heart valve 10, at the outflow end 14 of the prosthetic heart valve 10, or at both the inflow end 12 and the outflow end 14 of the prosthetic heart valve 10. Advantageously, the support frame at the inflow end 12 can be made rigid in order to provide more stability and improved functioning of the prosthetic heart valve 10. When a support frame is provided at both the inflow end 12 and the outflow end 14, the support frame at the inflow end 12 can be made more rigid than the support frame at the outflow end 14, or the support frames at both the inflow end 12 and outflow end 14 can be made with substantially similar rigidities.

The construction of the prosthetic heart valves 10 described in the present disclosure offers several advantages over conventional prosthetic heart valve designs. Generally, because the entire prosthetic heart valve (leaflets and root) can be produced from one continuous piece of biomaterial, failure points and stress concentrations that are associated with current prosthetic valves can be significantly reduced or otherwise eliminated. Further, the most vulnerable regions of the valve, the leaflet commissures, are generally eliminated and instead replaced with leaflets that gently fold inward and outward during each cardiac cycle with no areas of stress concentration. The vulnerable sutures are all located in the root behind the center of the leaflets, where minimal stress is located.

Furthermore, overlapping folds of the material or an appropriate suturing strategy during implantation can be used to accommodate somatic growth in pediatric patients. For example, the material can be secured to the native root rather than to itself at the adjacent folding sites to allow for circumferential expansion.

The foldable construction of the prosthetic heart valves described in the present disclosure allows for straightforward fabrication. The lower material demands also allow for a wider range of biocompatible materials, biomaterials, and/or tissues to be utilized in the construction of the prosthetic heart valve.

The prosthetic heart valves described in the present disclosure can be deployed using conventional surgical procedures, including both retrograde and antegrade approaches. In addition, the prosthetic heart valves described in the present disclosure can be deployed using minimally invasive procedures, such as a transcatheter aortic valve replacement (“TAVR”) procedure.

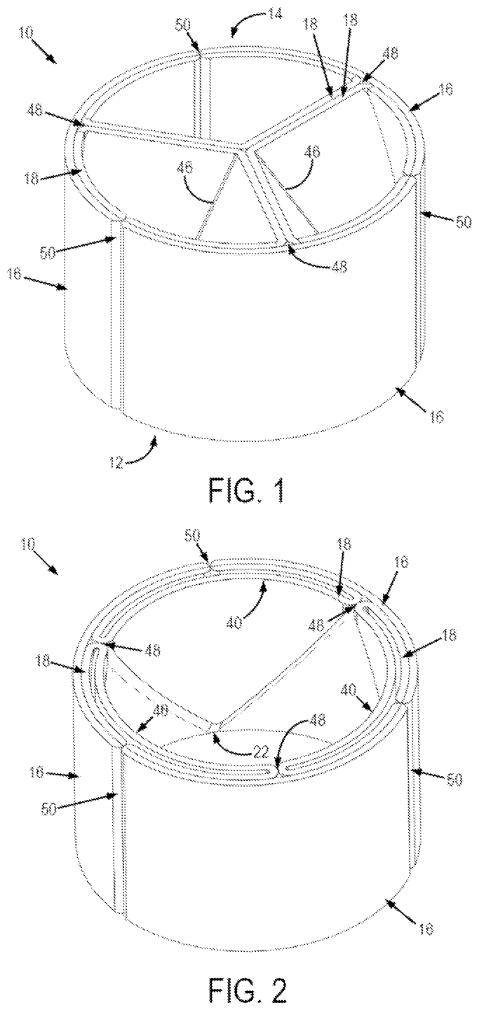

FIGS. 1 and 2 show an example of a prosthetic heart valve 10 according to some embodiments described in the present disclosure. FIG. 1 shows a prosthetic heart valve 10 in a closed state, and FIG. 2 shows the prosthetic heart valve 10 in an open state. The prosthetic heart valve 10 has a generally cylindrical, or otherwise tubular, shape that extends from an inflow end 12 to an outflow end 14. For example, as shown in FIGS. 1 and 2, the lower end of the prosthetic heart valve 10 may be an inflow end 12 and the upper end of the prosthetic heart valve 10 may be an outflow end 14, such that blood is able to flow from the inflow end 12 to the outflow end 14 when in use.

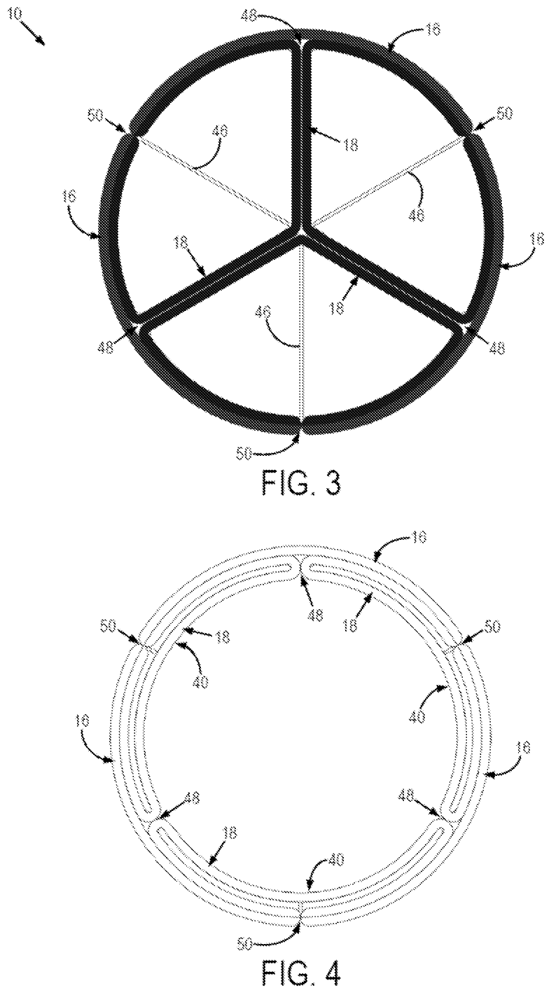

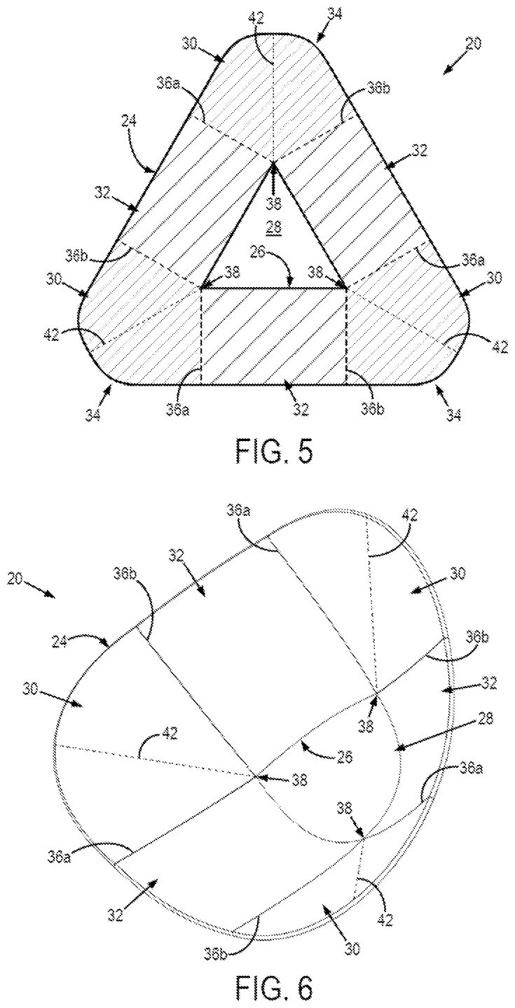

The prosthetic heart valve 10 generally includes a plurality of outer wall portions 16 and a plurality of leaflet portions 18. The outer wall portions 16 collectively define an outer wall, or frame, of the prosthetic heart valve 10, and the leaflet portions 18 collectively define leaflets, or cusps, of the prosthetic heart valve 10, as illustrated in FIGS. 3 and 4, which are top views of a prosthetic heart valve 10 in its closed state and open state, respectively. The outer wall portions 16 and leaflet portions 18 are defined by different folded portions of a single flexible substrate 20, such as the flexible substrate 20 shown in FIGS. 5 and 6, such that the prosthetic heart valve 10 is constructed from this single flexible substrate 20.

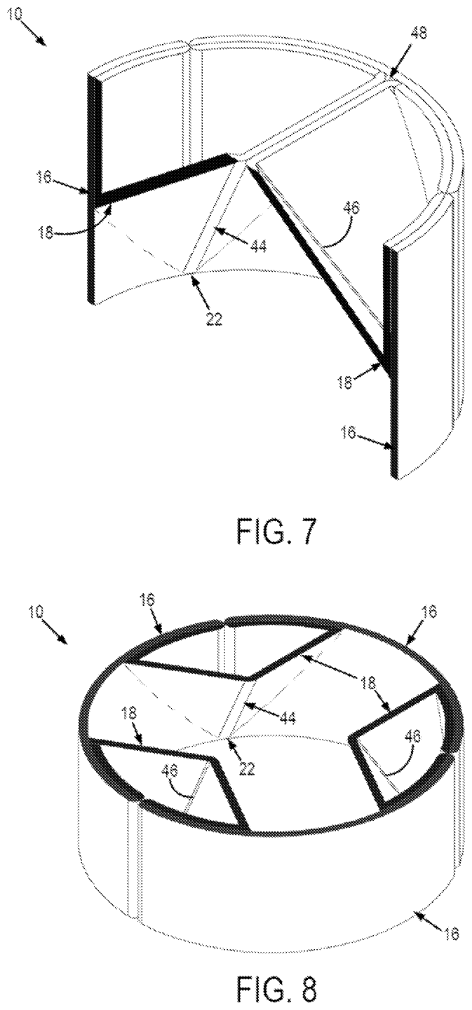

FIG. 7 shows a cross-sectional view of a prosthetic heart valve 10 taken in a plane that is parallel to the longitudinal axis of the prosthetic heart valve 10, and FIG. 8 shows a cross-sectional view of a prosthetic heart valve 10 taken in a plane that is transverse to the longitudinal axis of the prosthetic heart valve 10. The longitudinal axis of the prosthetic heart valve 10 may be defined as the central axis that extends through the center of the prosthetic heart valve 10 between the inflow end 12 and the outflow end 14 of the prosthetic heart valve 10. As such, the longitudinal axis may also be referred to as a flow axis of the prosthetic heart valve 10.

In FIGS. 7 and 8, the general shape of the leaflet portions 18 of the prosthetic heart valve 10 can be seen. The leaflet portions 18 generally define the leaflets, or cusps, of the prosthetic heart valve 10. Each leaflet portion 18 extends from a nadir point 22 at the inflow end 12 of the prosthetic heart valve 10 towards the free edge 40 of the leaflet portion 18, which resides in the transverse annular plane of the prosthetic heart valve 10. This transverse annular plane is transverse to the longitudinal axis and located at the outflow end 14 of the prosthetic heart valve 10.

The prosthetic heart valve 10 is generally operable as follows. When in the closed state, blood will flow into the central lumen of the prosthetic heart valve 10 at the inflow end 12. The blood will then impinge upon and thus apply pressure to the lower surface of the leaflet portions 18. The force of the blood on the leaflet portions 18 pushes the leaflet portions 18 outward to open the valve. Generally, the leaflet portions 18 are pressed outward from the flow axis and towards the outer wall portions 16 of the prosthetic heart valve 10. In this way, the prosthetic heart valve 10 is opened from its closed state shown in FIG. 1 to its open state shown in FIG. 2. Backpressure will then pull the leaflet portions 18 inward to close the valve and prevent retrograde flow. Generally, the leaflet portions 18 are pulled inward from the outer wall portions 16 towards the flow axis of the prosthetic heart valve 10. The prosthetic heart valves 10 described in the present disclosure thus have similar opening and closing mechanics to those of a native heart valve.

FIGS. 5 and 6 show examples of flexible substrates 20 that can be folded into a prosthetic heart valve 10, such as the prosthesis shown in FIGS. 1 and 2. The flexible substrate 20 shown in FIG. 5 is a generally flat substrate, whereas the flexible substrate shown in FIG. 6 is a curved substrate shaped as a generally hollow conical frustum. The flexible substrate 20 constitutes the prosthetic heart valve 10 in an unfolded configuration, whereas folding the flexible substrate 20 to create the prosthetic heart valve 10 constitutes the prosthetic heart valve 10 in a folded configuration.

The flexible substrate 20 may have a generally annular shape defined by an outer periphery 24 and an inner periphery 26 that circumscribes a central aperture 28 of the flexible substrate 20. As one example, the flexible substrate 20 can have a generally triangular annular shape, as shown in FIGS. 5 and 6. In other configurations, the flexible substrate 20 may have other shapes.

The flexible substrate 20 contains a plurality of leaflet-defining regions 30 and a plurality of wall-defining regions 32. When the flexible substrate 20 is folded into the folded configuration of the prosthetic heart valve 10, the leaflet-defining regions 30 form the leaflet portions 18 of the prosthetic heart valve 10. These leaflet portions 18 are shaped and operable as pliable heart valve leaflets, or cusps. Likewise, when the flexible substrate 20 is folded into the folded configuration of the prosthetic heart valve 10, the wall-defining regions 32 will form the outer wall portions 16 of the foldable heart valve 10.

In general, the flexible substrate 20 includes the same number of leaflet-defining regions 30 as wall-defining regions 32. As one non-limiting example, the foldable heart valve 10 can include three leaflet-defining regions 30 and three wall-defining regions 32. In other configurations, the flexible substrate 20 can contain more or fewer than three leaflet-defining regions 30 and/or wall-defining regions 32.

Each leaflet-defining region 30 is bounded by an arcuate segment 34 of the outer periphery 24 of the flexible substrate, a two edges 36 defined as line segments, which may be linear or arcuate segments, extending from the outer periphery 24 to the inner periphery 26. For instance one of the two edges 36a can extend from a first end of the arcuate segment 34 to a point on the inner periphery 26 of the flexible substrate 22. This point 38 may be, for example, a corner or vertex on the inner periphery 26. Similarly, the other of the two edges 36b can extend from the other end of the arcuate segment 34 to the same point 38 on the inner periphery 26. When the flexible substrate 20 is folded from the unfolded configuration to its folded configuration, thereby forming the prosthetic heart valve 10, these points 38 on the inner periphery 26 will correspond to the nadir points 22 of the respective leaflet portions 18.

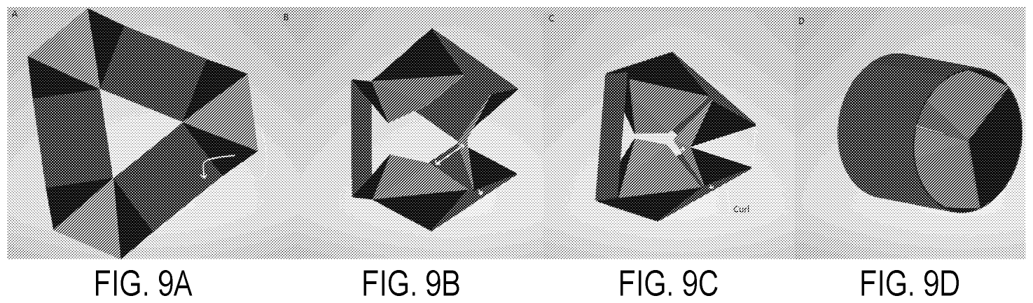

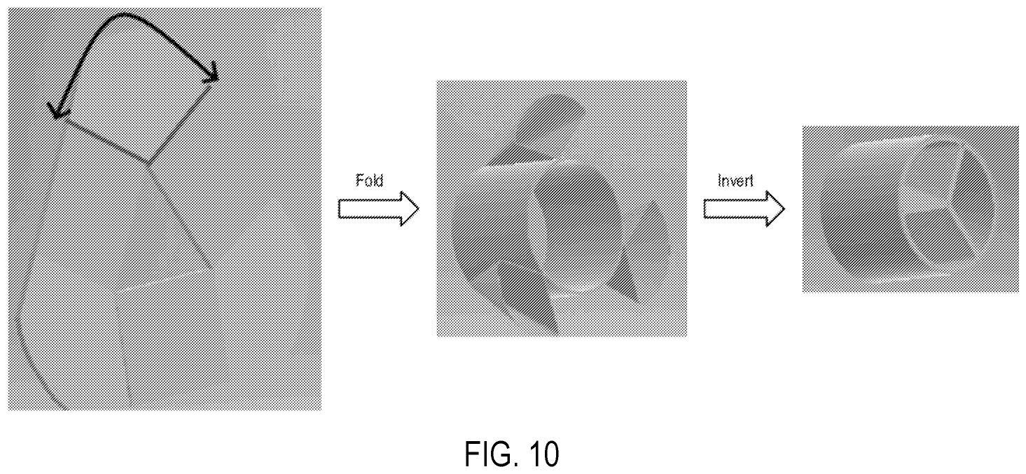

When folding the flexible substrate 20 from its unfolded configuration to its folded configuration, the leaflet-defining regions 30 can be folded along the fold line 42 while bringing edges 36a and 36b into contact with each other. In doing so, the flexible substrate 20 is further folded along each edge 36a and 36b such that the leaflet-defining regions 30 are folded towards the inner surface of the prosthetic heart valve 10 at each edge 36. Likewise, the wall-defining regions 32 are folded towards the outer surface of the prosthetic heart valve 10 at each edge 36. Further, folding the leaflet-defining regions 30 along the fold line 42 forms the central edge 44 of each leaflet portion 18 on its lower surface (i.e., the inflow side surface) and the central crease 46 of each leaflet portion 18 on its upper surface (i.e., the outflow side surface). The central edge 44 and/or central crease 46 of each leaflet portion 18 extends from the nadir point 22 at the inflow end 12 of the prosthetic heart valve 10 to the central point of the free edge 40 for a given leaflet portion 18. An example of this folding process is shown in FIGS. 9A-9D. In this example, FIG. 9A shows the initial pattern of the flexible substrate 20, and FIGS. 9B and 9C show transitional stages of the folding process. The fully folded prosthetic heart valve 10 is shown in FIG. 9D. The progression from FIG. 9A to FIG. 9B to FIG. 9C to FIG. 9D is shown by representative arrows.

The region where the end of the free edge 40 of one leaflet region 18 abuts the end of the free edge 40 of an adjacent leaflet region 18 defines a commissure 48. Advantageously, the commissures 48 of the prosthetic heart valves 10 described in the present disclosure do not experience high stresses based on the simple, folded construction of the prosthetic heart valve 10.

The region where edges 36 and 36b are brought into contact will form a seam 50 where the adjacent leaflet portions 18 and outer wall portions 16 can be coupled together in order to form the functional prosthetic heart valve 10. For instance, the seams can be joined or otherwise coupled via adhesive, sutures, or the like. Advantageously, this construction allows for vulnerable sutures to be located behind the leaflet portions 18 of the prosthetic heart valve 10 and, thus, away from stresses. As an added advantage, by using two suture lines along the opposed vertical edges of the seam 50 and directly to the native root, circumferential expansion of the prosthetic heart valve 10 is capable, thereby accommodating somatic growth of a pediatric heart. This advantage can be realized even if the valve material is inert because the shape changes to accommodate the expanding annulus. A tissue engineered (i.e., living) valve with this design may also allow the prosthetic heart valve 10 itself to grow along with the expanding annulus.

In some instances, the flexible substrate 20 can be folded in such a way that the leaflet-defining regions 30 are folded towards the inner lumen of the prosthetic heart valve 10 when in its folded configuration. In other instances, the flexible substrate 20 can be folded in such a way that the leaflet-defining regions 30 will be on the outside of the central lumen of the prosthetic heart valve 10. The prosthetic heart valve 10 can then be inverted, such that the leaflet portions 18 will be contained within the central lumen of the prosthetic heart valve 10. An example of this folding and inversion is shown in FIG.

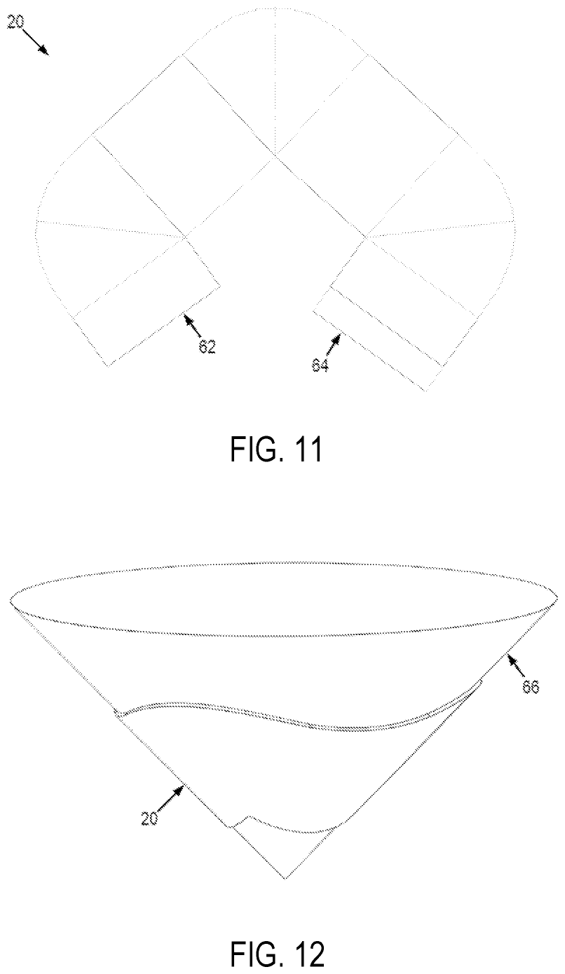

Although the flexible substrates 20 shown in FIGS. 5 and 6 are a continuous annular substrate, in some configurations the flexible substrate can be manufactured as a discontinuous annular substrate, such as the flexible substrate 20 shown in FIG. 11. In this instance, the flexible substrate 20 can be formed as a flat substrate having a first cut end 62 and a second cut end 64. The first cut end 62 can be brought into contact with and coupled to the second cut end 64 to form a continuous annular shape. For instance, when the first cut end 62 is brought into contact with and coupled to the second cut end 64, the flexible substrate can form an annular shape having a curved surface, similar to the flexible substrate 20 illustrated in FIG. 5.

In this way, the flexible substrate 20 can be manufactured as a flat substrate, which may simplify the manufacturing process, and then made into an annular shape with a curved surface to facilitate folding of the flexible substrate 20 into the prosthetic heart valve 10. For instance, the flexible substrate 20 can be easily manufactured by cutting a flat sheet of material according to a pattern that defines the shape of the flexible substrate 20. The two cut ends 52 and 54 of the flexible substrate 20 can then be joined (e.g., via adhesive, sutures, or otherwise) in order to form an annular shape with a curved surface.

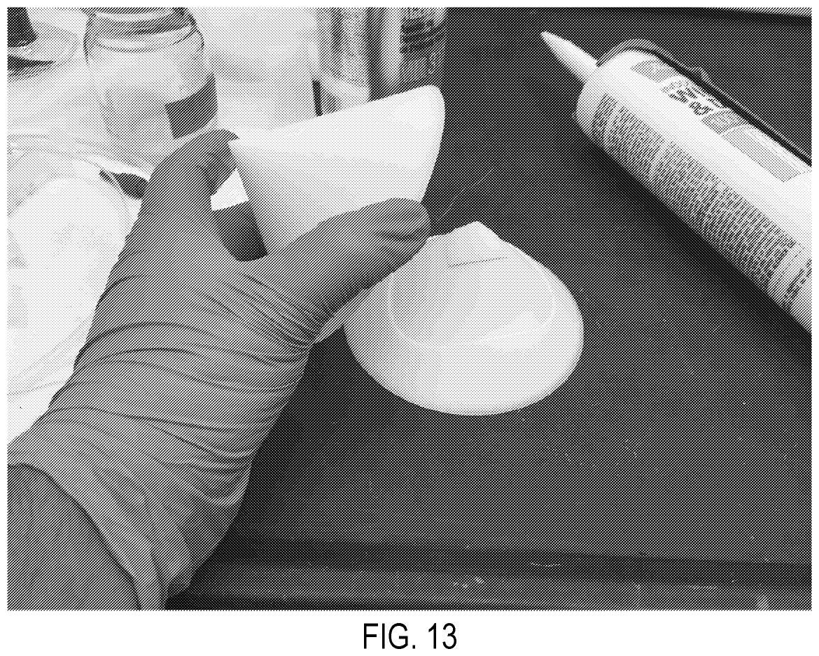

Alternatively, the flexible substrate 20 can be manufactured as an annular shape with a curved surface. As one example, the flexible substrate 20 can be manufactured on the surface of an appropriately shaped form, mold, or the like. For instance, as shown in FIG. 12, the flexible substrate 20 can be manufactured on the surface of a conical mold 66, such that the resulting flexible substrate 20 will have a continuous annular shape with a generally curved surface. An example of such a conical mold 66 is shown in FIG. 13. This example conical mold 66 was constructed using an additive manufacturing process, such as 3D printing. In still other instances, the flexible substrate can be cut from a conical sheet of material according to a pattern that defines the desired shape of the flexible substrate 20.

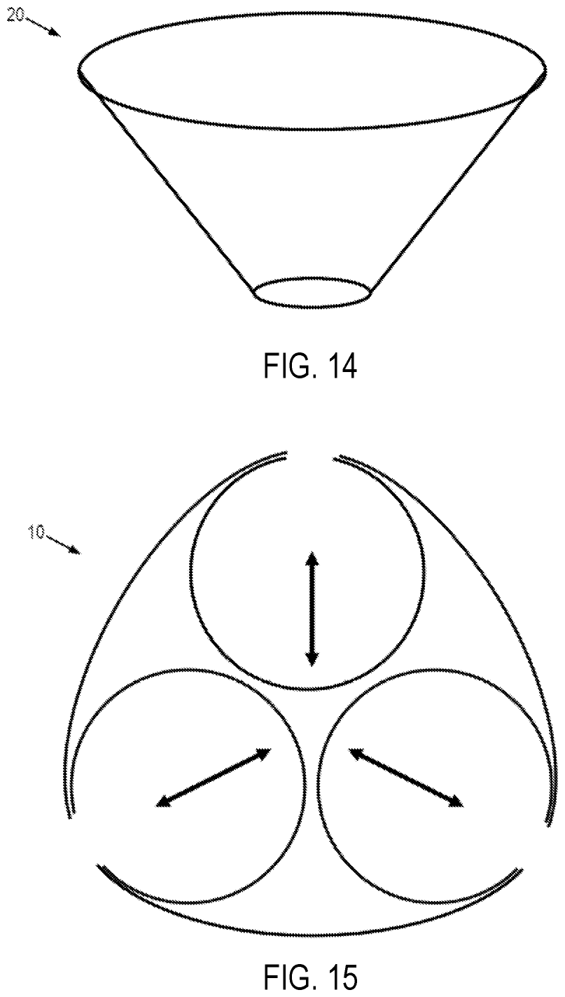

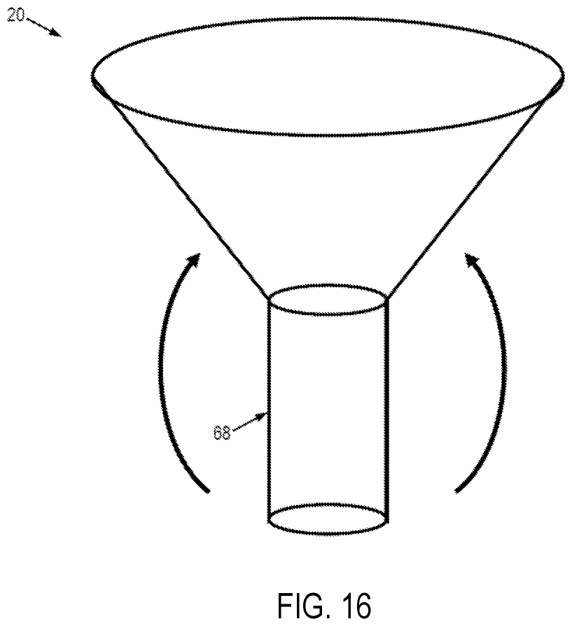

In some alternative configurations, the flexible substrate 20 can have a generally conical annular shape, such as shown in FIG. 14. This flexible substrate 20 can then be folded inward at three locations, as shown in FIG. 15, in order to form three conical leaflets, or cusps, within a cylindrical outer frame. These leaflets can flex inward and outward to form a functional prosthetic heart valve 10. In a modified configuration, shown in FIG. 16, a tubular extension 68 can be added onto the bottom of the conical flexible substrate 20. After the three leaflets are folded inward, the tubular extension 68 can be inverted upwards to hold the folded shape of the prosthetic heart valve 10, effectively functioning as the outer wall, or frame, of the prosthesis. This has the advantage of easy assembly with no seams and no sutures.

When constructed for use as an aortic heart valve, the prosthetic heart valve 10 can be modified to include openings for the coronary ostia. For example, holes can be made in the outer wall portions 16 and/or outer surface of the leaflet portions 18 such that when the prosthetic heart valve 10 is deployed the holes will align with the coronary ostia.

In some configurations, a sewing cuff and/or extension can be added on to the prosthetic heart valve 10 to allow for suturing above and/or below the prosthetic heart valve 10.

The flexible substrate 20 can be composed of any suitable biocompatible material, biomaterial, engineered tissue, and/or natural tissue that can be formed into a flat sheet or around a form, such as a conical frustum. Because of the ease of fabrication and less demanding durability requirements of the prosthetic heart valves described in the present disclosure, the types of materials that can be used to make the valves can be greatly expanded relative to other prosthetic heart valve designs. For example, tissue engineering approaches can be implemented, whereby living materials capable of remodeling, repair, and growth are used to fabricate the valve. Similarly, blood compatible materials that do not require lifelong compliance with anticoagulation therapy can also be used. As non-limiting examples, blood compatible materials such as polydimethylsiloxane, expanded polytetrafluoroethylene, polyethylene terephthalate, and/or polyurethane can be used.

The material type, flexible substrate thickness, and other dimensions can be adjusted in order to fine tune or otherwise alter the valve mechanics and/or functionality. For example, in some embodiments the flexible substrate 20 can have a thickness of not more than 0.6 mm. As one advantageous example, the flexible substrate can have a thickness of 0.4 mm. In other examples, the flexible substrate 20 can have a thickness in a range of about 0.1 mm to about 0.6 mm (e.g., 0.1 mm, 0.15 mm, 0.2 mm, mm, 0.3 mm, 0.35 mm, 0.4 mm, 0.45 mm, 0.5 mm, 0.55 mm, or 0.6 mm, or other thicknesses in the range of about 0.1 mm to about 0.6 mm). In general, as the flexible substrate 20 is made thicker, a less stiff material can be used, and as the flexible substrate is made thinner, a more stiff material can be used.

As one example, the flexible substrate 20 can be composed of biocompatible materials, such as vinyl, nylon, polydimethylsiloxane, expanded polytetrafluoroethylene, polyethylene terephthalate, polyethylene glycol, poly(lactic acid), poly(glycolic acid), poly(lactic-co-glycolic acid), polycaprolactone, poly(glycerol sebacate), or polyurethane.

As another example, the flexible substrate 20 can be composed of biomaterials, such as gelatin, collagen, elastin, alginate, collagen gel. Biomaterials may include tissue-engineered materials, such as cells on a scaffold, or decellularized membranes and/or polymers. For instance, decellularized porcine small intestinal submucosa, decellularized porcine pericardium, decellularized bovine pericardium, and/or decellularized amniotic membrane can be used.

As still another example, the flexible substrate 20 can be composed of tissues, such as bovine pericardium, porcine pericardium, small intestinal submucosa, amniotic membrane.

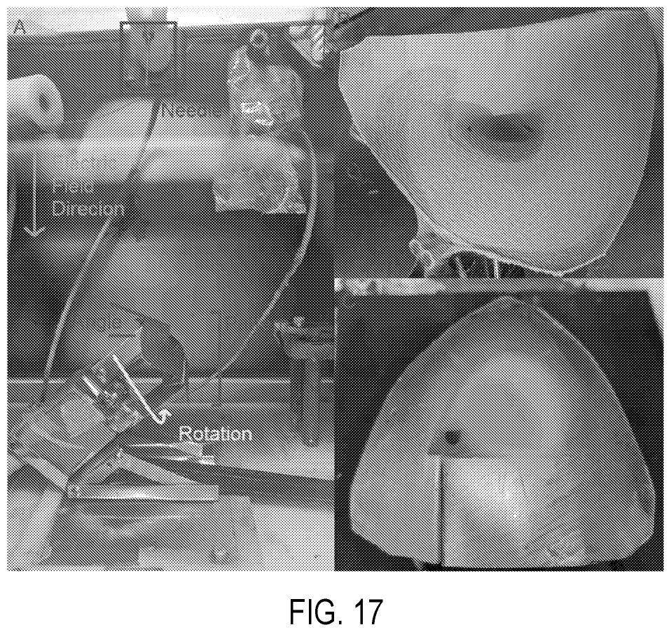

In one non-limiting example, the flexible substrate 20, and thus the resulting prosthetic heart valve 20, can be composed of electrospun polyurethane. Referring now to FIG. 17, in one example the flexible substrate 20 can be fabricated from electrospun nanofibers of polyurethane using a rotating collector. In this example, polyurethane was dissolved in dimethyl-acetamide (“DMA”) to form a 15% (w/v) solution. The 15% PU in DMA is then emitted from the needle and pulled by an electric field onto the grounded metal pattern using a downward electric field. As one non-limiting example, the collector angle can be set to 12 degrees above the horizontal and the rotation speed of the rotating pattern can be set to 3 RPM, in order to achieve a relatively uniform material. The resulting flexible substrate 20 can be fabricated such that the polyurethane fibers are aligned, or such that they are generally unaligned.

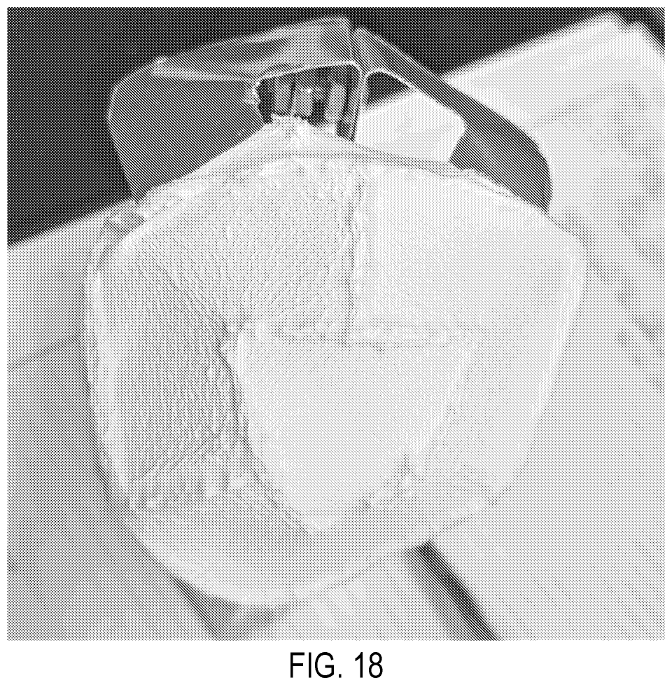

Advantageously, when using an electrospinning process the resulting flexible substrate can be made thicker in regions that correspond to the free edge 40 of the leaflet portions 18 when the flexible substrate 20 is folded into the folded configuration of the prosthetic heart valve 10, as shown in FIG. 18. In this way, the prosthetic heart valve 10 can be fabricated in a way that mimics native leaflet, or cusp, morphology (i.e., the thickened lunula), which can improve the mechanics and/or functionality of the prosthetic heart valve 10. For example, having a thicker free edge 40 can provide for better control over the functionality of the prosthetic heart valve 10.

The present disclosure has described one or more preferred embodiments, and it should be appreciated that many equivalents, alternatives, variations, and modifications, aside from those expressly stated, are possible and within the scope of the invention.

1. A prosthetic heart valve, comprising:
a flexible substrate having a plurality of leaflet-defining regions and a plurality of outer wall-defining regions, such that when the flexible membrane is folded into a folded configuration the plurality of outer wall-defining regions form a cylindrical outer wall and the plurality of leaflet-defining regions form a corresponding plurality of flexible leaflets arranged within a lumen of the cylindrical outer wall.

2. The prosthetic heart valve of claim 1, wherein the flexible substrate has a flat annular shape.

3. The prosthetic heart valve of claim 2, wherein the annular shape is a triangular annular shape.

4. The prosthetic heart valve of claim 3, wherein the triangular annular shape has rounded corners on its outer periphery.

5. The prosthetic heart valve of claim 1, wherein the flexible substrate has a curved annular shape.

6. The prosthetic heart valve of claim 5, wherein the flexible substrate has a conical frustum annular shape.

7. The prosthetic heart valve of claim 1, wherein the flexible substrate is composed of a biocompatible material.

8. The prosthetic heart valve of claim 7, wherein the biocompatible material comprises electrospun polyurethane.

9. The prosthetic heart valve of claim 1, wherein the flexible substrate is composed of a biomaterial.

10. The prosthetic heart valve of claim 9, wherein the biomaterial is a decellularized membrane.

11. The prosthetic heart valve of claim 1, wherein the flexible substrate is composed of a tissue.

12. The prosthetic heart valve of claim 1, wherein the flexible substrate has a an flat annular shape that is discontinuous at a cut region defined by opposing first and second cut edges of the flexible substrate, wherein when the first cut edge is coupled to the second cut edge the flexible substrate forms a curved annular shape.

13. A prosthetic heart valve device, comprising:
a flexible substrate having an annular shape comprising:
an outer periphery that circumscribes the flexible substrate;
an inner periphery that defines an aperture of the annular shape;
a plurality of leaflet-defining regions, each defined by:
an arcuate segment extending along the outer periphery from a first point to a second point;
a first edge line extending from the first point to a nadir point on the inner periphery;
a second edge line extending from the second point to the nadir point on the inner periphery;
a plurality of wall-defining regions, each defined as a portion of the flexible substrate opposed by the first edge line of a first leaflet-defining region and the second edge line of a second leaflet-defining region that is adjacent the first leaflet-defining region;
wherein when in a folded configuration the first edge line and the second edge line of each leaflet-defining portion meet in a seam region such that the plurality of wall-defining regions circumscribe a cylindrical volume and define an outer wall, and such that each leaflet-defining region extends from its nadir point at an inflow end of cylindrical volume to at least a free edge lying in a transverse plane at an outflow end of the cylindrical volume.
